(19) United States
(12) Patent Application Publication
(10) Pub. No.: US 2011/0020417 A1
(43) Pub. Date: Jan. 27, 2011

(54) CHEWING GUM

(75) Inventors: Aya Takeyama, Tokyo (JP); Makoto Sato, Minamisaitama-gun (JP); Masahiro Tani, Saitama-shi (JP)

Correspondence Address: MILES & STOCKBRIDGE PC, 1751 PINNACLE DRIVE, SUITE 500, MCLEAN, VA, 22102-3833, US

(73) Assignee: LOTTE CO., LTD, TOKYO (JP)

(21) Appl. No.: 12/919,218

(22) PCT Filed: Feb. 17, 2009
(86) PCT No.: PCT/JP2009/053121

§ 371 Date: Aug. 25, 2010

(30) Foreign Application Priority Data
Feb. 25, 2008 (JP) 2008-043584

(51) Int. Cl. A61K 8/02; A61K 8/34; A61Q 11/00

(52) U.S. Cl. 424/401; 424/48

(57) ABSTRACT

A novel chewing gum is provided which gives a strong coolness feeling and excellent mouth feeing in chewing. The chewing gum contains particles of one or more of crystalline matter selected from the group consisting of erythritol, xylitol, and sorbitol in an amount ranging from 5% to 70% by weight based on the entire weight of the chewing gum.

### TECHNICAL FIELD

The present invention relates to chewing gum.

### BACKGROUND ART

Conventionally, erythritol which is highly anticarious is added as an anticaries agent to foodstuffs, toothpastes, mouth-cleaning solutions, and the like (Japanese Patent No. 3459334, Japanese Patent Publication No. S56-018180).

Erythritol is also useful as a sweetening agent for foods. Japanese Patent No. 3312028 discloses a chewing gum having a high anti-caries property, which contains a sweetening agent composed of a mixture of erythritol (70 wt %) and a liquid sugar or a sugar alcohol. Japanese Patent Publication No. H07-028671 discloses a process for producing a chewing gum containing crystalline meso-erythritol having a crystal particle size of 50 μm and less to reduce a feeling rough just like biting sands in chewing the gum containing a powdery sugar having a larger particle size.

On the other hand, a sugar alcohol which generates a large quantity of dissolution heat is blended into a chewing gum to cause a coolness feeling in chewing the gum. However, the sugar alcohol added to the conventional chewing gum is powdery, having a particle size of less than 0.1 mm. Such a powdery sugar alcohol cannot create a strong coolness feeling by its endothermic reaction. Therefore how to make the chewing gum to present the heat absorption has been a problem.

### DISCLOSURE OF THE INVENTION

The present invention intends to provide a chewing gum which gives a strong feeling of coolness and texture in chewing the gum.

The chewing gum of the present invention contains crystalline matter of at least one of the substances selected from the group of erythritol, xylitol, and sorbitol (hereinafter referred to simply as “a crystalline matter”) in an amount of 5% to 70% by weight of the entire chewing gum. The particle size of the crystalline matter may range from 0.1 mm to 2 mm. The main body of the chewing gum stick may be coated.

The present invention is thus comprised of the crystalline matter and as a result creates the strong coolness feeling and causes a good oral feeling when eating.

### BRIEF DESCRIPTION OF THE DRAWING

FIG. 1 is a perspective view of a partially cutaway chewing gum stick of an example of the present invention.

### BEST MODE FOR CARRYING OUT THE INVENTION

The chewing gum of the present invention contains a crystalline matter in an amount of 5% to 70% by weight of the entire chewing gum. With the crystalline matter blended at a content of less than 5 wt %, the intended strong coolness feeling of the chewing gum will not be achieved in comparison with the conventional powder-containing chewing gum. The blending ratio of the crystalline matter of higher than 70 wt % is not preferred since, at a higher blending ratio, the associative strength of the entire gum can be lost to cause pulverization of the blended gum in the production process to cause a defective product, or the chewing gum can be destroyed into powdery particles during chewing in the mouth.

The crystalline matter has preferably a crystal particle size ranging from 0.1 mm to 2 mm. At a particle size of the crystalline matter of less than 0.1 mm, the coolness feeling given is not significantly different from that of the gum containing powdery sugar. On the other hand, at a particle size of the crystalline matter of larger than 2 mm, the associative strength is poor, and the crystalline matter is excessively hard to cause a foreign matter feeling or a discomfort feeling. Incidentally, the crystal size of 2 mm corresponds to the size of commercial coarse sugar particles.

The sweetening agent other than the above-mentioned crystalline matter blended into the chewing gum is not limited specially, and includes monosaccharides, disaccharides, oligosaccharides, sugar alcohols, and strong sweetening agents. Such a sweetening agent may be used singly or in combination of two or more thereof.

The gum base may be constituted of any material which is used for a conventional chewing gum. The gum base may be composed of a single component or in combination of two or more thereof.

The flavor and the colorant to be added to the gum may be any material which is used for foodstuffs. The flavor may be a composition or a simple substance, and may be used singly or in combination thereof. The colorant may be a natural colorant or a synthetic colorant, and the amount of addition and the colorant combination are not limited.

A supplemental additive may be blended, such as a sour agent, for imparting a taste. Any sour agent may be used singly or in combination without a special limitation. Another additive such as a thickener or a softening agent may be used like in conventional chewing gum.

The shape of the chewing gum of the present invention is not limited. The chewing gum is molded into various shapes by a molding apparatus such as an extruder, a filling apparatus, a cutting apparatus (cutter), and a mold. The molding apparatus may be used singly or in combination of two or more thereof.

Coating of the gum is not limited in the present invention, and the gum may be coated suitably as necessary. The process for the coating can be conducted by a conventional method without employing a special method. The ratio of the coating is not specially limited, preferably ranges from 10% to 100% by weight, more preferably from 20% to 60% by weight based on the center gum.

The present invention is described specifically with reference to test results and examples without limiting the invention. In the formulations shown below, the weight ratio is based on the weight of the entire chewing gum unless otherwise mentioned. The crystalline matter is sieved with a 16-mesh sieve (opening of 1 mm), and the same kind of gum base is used in the examples unless otherwise mentioned.

The evaluation is made for “coolness feeling” and “texture”. The coolness feeling is evaluated on four grades with improvement in comparison with chewing gum A as the control: A, improved remarkably; B, improved moderately; C, improved slightly; D, not improved.

The texture is evaluated on four grades with the extent of good crisp texture (brittleness feeling) in chewing the sample gum in comparison with chewing gum A as the control: A, crunch being felt remarkably; B, crunch being felt moderately; C, crunch being felt slightly; D, crunch being not felt.

### Test Example 1

This test was conducted to find the strong coolness feeling and good crisp texture of the chewing gum achieved by blending the crystalline matter of erythritol, xylitol, or sorbitol in comparison with the sample containing the crystal powder thereof.

### 1. Preparation of Samples:

26 parts of powdery xylitol, 40 parts of crystalline sugar, 30 parts of a gum base, 2 parts of an flavor, and 2 parts of a softening agent were blended enough for 15 to 30 minutes by a chewing-gum mixer. This blended matter was rolled into a shape of a plate of 3 grams. The sensory evaluation was conducted after left standing for 12 hours to one day.

Samples 1 to 5 were prepared by changing the kind of the crystalline sugar as shown in Table 1. Chewing gum A as a control sample was prepared in the same manner.

### 2. Test Method:

Samples 1-5 were evaluated sensorily in comparison with Chewing-gum A by well-trained expert panel members. Table 1 shows the evaluation results.

TABLE 1
TESTS ON KINDS OF CRYSTALLINE SUGAR

| | | Sample 1 | Sample 2 | Sample 3 | Sample 4 | Sample 5 |
|---|---|---|---|---|---|---|
| Kind of crystal | Gum A | Cane Sugar | Glucose | Erythritol | Xylitol | Sorbitol |
| Gum Base | 30 | 30 | 30 | 30 | 30 | 30 |
| Powdery Xylitol | 66 | 26 | 26 | 26 | 26 | 26 |
| Sugar (Crystal) | | 40 | 40 | 40 | 40 | 40 |
| Flavor | 2 | 2 | 2 | 2 | 2 | 2 |
| Softening Agent | 2 | 2 | 2 | 2 | 2 | 2 |
| Total | 100 | 100 | 100 | 100 | 100 | 100 |
| Sensory Evaluation | D | D | D | A | A-B | A-B |
| (Coolness Feeling) | | | | | | |
| Sensory Evaluation | D | A | C | A | A | B |
| (Texture) | | | | | | |

### 3. Test Results:

Table 1 shows the evaluation results. The sample employing the crystalline erythritol is excellent in coolness feeling as well as in good crisp texture in chewing, and is highly suitable for carrying out the present invention. Further, the samples employing xylitol or sorbitol, are somewhat inferior to the sample employing erythritol, but gives high good crisp texture to be suitable for use.

### Test Example 2

This test was conducted to find dependence of the coolness feeling and good crisp texture in chewing of the gum during chewing on the amount of the crystalline sugar blended into the chewing gum.

### 1. Preparation of Samples:

0-70 parts of Powdery xylitol, 5-75 parts of crystalline erythritol, 21 parts of a gum base, 2 parts of an flavor, and 2 parts of a softening agent were blended enough for 15 to 30 minutes by a chewing-gum mixer. This blended matter was rolled into a shape of a plate of 3 grams. The sensory evaluation was made after left standing for 12 hours to one day.

Samples 6 to 11 were prepared by changing the amount of the crystalline erythritol as shown in Table 2.

### 2. Test Method:

Samples 6-11 were evaluated sensorily in comparison with Chewing-gum A prepared in Example 1 by well-trained expert panel members. Table 2 shows the evaluation results.

TABLE 2
TESTS ON AMOUNT OF CRYSTALLINE SUGAR

| | Sample 6 | Sample 7 | Sample 8 | Sample 9 | Sample 10 | Sample 11 |
|---|---|---|---|---|---|---|
| Gum Base | 21 | 21 | 21 | 21 | 21 | 21 |
| Powdery Xylitol | 72 | 70 | 65 | 25 | 5 | 0 |
| Crystalline Erythritol | 3 | 5 | 10 | 50 | 70 | 75 |
| Flavor | 2 | 2 | 2 | 2 | 2 | 2 |
| Softening Agent | 2 | 2 | 2 | 2 | 2 | 2 |
| Total | 100 | 100 | 100 | 100 | 100 | 100 |
| Sensory Evaluation | D | B | A | A | A | — (*2) |
| (Coolness Feeling) | | | | | | |
| Sensory Evaluation | D | A | A | A | B (*1) | — (*2) |
| (Texture) | | | | | | |

(*1): The texture of Sample 10 is evaluated as Grade B, since “the texture is remarkably improved from that of Gum A but it gives a rough crisp texture of the crystal particles”.
(*2): Sample 11 became powdery in the blending operation, and could not be shaped into a gum stick.

### 3. Test Result:

Table 2 shows the evaluation results. Strong coolness feeling and crisp texture in chewing were achieved at the content of the crystalline erythritol in the chewing gum ranging from 5 to 70 parts, the coolness feeling and crisp texture were very weak at the content of crystalline erythritol of less than 5 parts, and the associative strength of the chewing gum was poor and the gum became powdery without forming a lump at the content of the crystalline erythritol of higher than 70 parts. Further, even at a crystal blending ratio within the range in which the gum can be prepared, at a high crystal content, the texture caused was crunchy such that the crystal particles are directly chewed, and was evaluated to be not excellent. From this tendency in the above results, the content of the crystalline erythritol is preferably in the range from 5 to 70 parts, more preferably from 10 to 50 parts.

### Test Example 3

This test was conducted to find dependence of the coolness feeling and crisp texture during chewing of the chewing gum on the particle size of the crystalline matter blended in the chewing gum.

### 1. Preparation of Samples:

35 parts of powdery xylitol, 40 parts of crystalline erythritol, 21 parts of a gum base, 2 parts of an flavor, and 2 parts of a softening agent are blended enough for 15 to 30 minutes by a chewing-gum mixer. This blended matter was rolled into a shape of a plate of 3 grams. The sensory evaluation was made after left standing for 12 hours to one day.

Samples 12 to 16 were prepared by changing the particle size of the crystalline erythritol as shown in Table 3.

### 2. Test Method:

Samples 12-16 were evaluated sensorily in comparison with Chewing-gum A prepared in Example 1 by well-trained expert panel members. Table 3 shows the evaluation results.

TABLE 3
TESTS ON CRYSTAL PARTICLE SIZE

| | Sample 12 | Sample 13 | Sample 14 | Sample 15 | Sample 16 |
|---|---|---|---|---|---|
| Crystal Particle Size (mm) | 0.08 | 0.1 | 1.5 | 2.0 | 2.2 |
| Gum Base | 21 | 21 | 21 | 21 | 21 |
| Powdery Xylitol | 35 | 35 | 35 | 35 | 35 |
| Crystalline Erythritol | 40 | 40 | 40 | 40 | 40 |
| Flavor | 2 | 2 | 2 | 2 | 2 |
| Softening Agent | 2 | 2 | 2 | 2 | 2 |
| Total | 100 | 100 | 100 | 100 | 100 |
| Sensory Evaluation | D | B-C | A | A | B |
| (Coolness Feeling) | | | | | |
| Sensory Evaluation | D | B | A | B-C | D |
| (Texture) | | | | | |

### 3. Test Results:

Table 3 shows the test results. The crystalline erythritol having a particle size of less than 0.1 mm blended in the chewing gum did not give the coolness feeling and crisp texture. In contrast, the crystalline erythritol having a particle size larger than 2 mm gave coolness feeling but tended to be weaker than that of smaller than 2 mm. Further, it gave a rough crispness feeling just like biting a fragile foreign matter to be evaluated to be of a low grade. From the above results, for significant coolness feeling and significant crisp texture, the particle size of the blended crystal ranges preferably from 0.1 to 2 mm, more preferably in a size of about 1 mm.

### Example

Based on the above three Test Examples, an Example is described below under optimum conditions in the present invention.

In this Example, erythritol was used as the crystalline matter. The crystalline matter had a particle size of 1 mm, and was blended in a ratio of 17 parts.

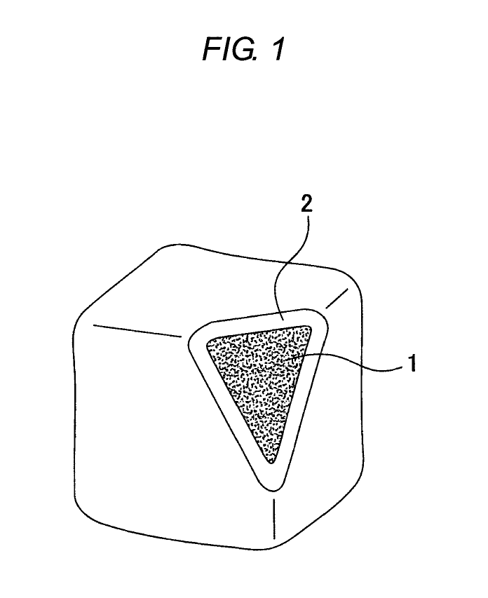

Table 4 shows the formulation of the chewing gum of this Example. The chewing gum in this Example was prepared by a conventional chewing gum production process without employing a special method. Specifically the chewing gum was prepared through the steps as follows: (1) input of the chewing gum source materials (including the crystalline matter and other materials) simultaneously, (2) blending by a mixer, (3) extrusion (by an extruder), (4) rolling, (5) molding, and (6) coating. After the blending, the chewing gum was rolled and molded into a cube having sides of about 11 mm, and then it was coated. The coating was conducted by a conventional chewing gum coating process. The coating formulation contains maltitol as the main component, a brightening agent, and other additives. FIG. 1 is a perspective view of the partially cutaway chewing gum stick of this Example.

TABLE 4
FORMULATION OF CHEWING GUM OF EXAMPLE

| | |
|---|---|
| Gum Base | 23 |
| Sugar (excluding Crystalline Erythritol) | 56 |
| Crystalline Erythritol | 17 |
| Colorant | 0.1 |
| Flavor | 2.1 |
| Supplemental Additive | 1.8 |
| Total | 100 |

The chewing gum of this Example contains crystalline erythritol of the particle size of 1 mm in an amount of about 17% by weight based on the total weight of the chewing gum as described above. Therefore the chewing gum gave a strong coolness feeling in chewing the gum in comparison with a conventional chewing gum containing a powdery sugar alcohol only. Further it gave good crisp texture which cannot achieved by a conventional chewing gum. Similar effects were achieved by blending the crystalline matter in an amount ranging from 5% to 70% by weight based on the total weight of the chewing gum.

Similar effects were achieved by blending crystalline xylitol or crystalline sorbitol in place of the crystalline erythritol.

The present invention has been described with reference to exemplary embodiments without limiting the technical range of the present invention. The present invention can be carried out in various modifications without departure from the technical idea or main feature of the present invention.

This application claims the benefit of Japanese Patent Application No. 2008-043584, filed Feb. 25, 2008, which is hereby incorporated by reference herein in its entirety.

1. A chewing gum containing particles of one or more of crystalline matter selected from the group consisting of erythritol, xylitol, and sorbitol in an amount ranging from 5% to 70% by weight based on the entire weight of the chewing gum.

2. The chewing gum according to claim 1, wherein the size of the particles of the crystalline matter ranges from 0.1 mm to 2 mm.

3. The chewing gum according to claim 1, wherein the main body of the chewing gum is coated.

4. The chewing gum according to claim 2, wherein the main body of the chewing gum is coated.
